(19) United States
(12) Patent Application Publication
(10) Pub. No.: US 2006/0183973 A1
(43) Pub. Date: Aug. 17, 2006

(54) Endoscopic devices and method of use

(76) Inventor: Michael M. Kamrava, Beverly Hills, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/388,467

(22) Filed: Mar. 24, 2006

Related U.S. Application Data
(63) Continuation-in-part of application No. 10/725,623, filed on Dec. 1, 2003.
Application No. 10/725,623 is a continuation-in-part of application No. 10/080,177, filed on Feb. 19, 2002.
Application No. 10/080,177 is a continuation-in-part of application No. 09/759,415, filed on Jan. 12, 2001, now Pat. No. 6,623,422.

(51) Int. Cl. A61B 1/00; A61B 1/04

(52) U.S. Cl. 600/105; 600/104; 600/114

(57) ABSTRACT

A catheter including a shaft including a body with a proximal portion and a distal portion, the body defining an opening from the proximal portion to the distal portion, the distal portion having an exterior dimension suitable for insertion into a body of a subject as a procedural instrument, the distal portion having an end that is beveled in a first direction across an end opening, such that a length of the shaft to a first point is a first length and a length of the shaft to a second point on the end is a second length longer than the first length, a portion of the shaft extending from the second point defining a tip, wherein the tip comprises a material that has sufficient rigidity to penetrate an endometrial lining of a subject and sufficient flexibility to resist penetration of a uterine muscle of a subject.

### CROSS-REFERENCE TO RELATED APPLICATION

This application is a continuation-in-part of co-pending U.S. patent application Ser. No. 10/725,623, filed Dec. 1, 2003 which is a continuation-in-part of co-pending U.S. patent application Ser. No. 10/080,177, filed Feb. 19, 2002, and which is a continuation-in-part of U.S. patent application Ser. No. 09/759,415, issued as U.S. Pat. No. 6,623,422 on Sep. 23, 2003.

### BACKGROUND

1. Field

The embodiments disclosed herein relate generally to endoscopic devices, including hysteroscopes and related devices for microsurgical use.

2. Description of Related Art

Improving the success of in vitro fertilization (IVF) depends on many factors, one of which is the delivery or transfer of the embryo to the endometrial lining of the uterus and the successful implantation of the embryo therein. It is well known in the art that assisting an embryo to adhere to, or implant within, a predetermined area of the endometrial lining of the uterine wall, as opposed to simply releasing the embryo into the uterus, will enhance the success of IVF.

One method of assisted embryo transfer is found in U.S. Pat. No. 6,010,448 to Thompson in which an embryo is transferred with the aid of an endoscopic device, via a flexible catheter, to the endometrial lining and affixed thereto with an adhesive.

Another method of embryo transfer is taught in U.S. Pat. No. 5,360,389 to Chenette in which, after using pressurized CO₂ gas to distend the uterine walls, an endoscope is used to select an implantation site. A catheter is then used to forcibly inject the embryos into the endometrial lining.

While the embryo transfer methods of these prior art types may be generally satisfactory for their intended purposes, implantation problems can arise in which the trauma to the delicate embryos by either an injection or “adhesion” may yield less than optimal solutions and fail to achieve high IVF success rates. Accordingly, improved devices that may be useful, in one aspect, in intrauterine procedures such as IVF are desired. An improved embryo transfer method is also desired.

### SUMMARY

A catheter, an endoscope (hysteroscope), and a method of introducing at least one embryo into a uterus of a subject is described. One object of the device(s) and/or method is to provide a simple gentle method for intrauterine procedures such as embryo transfer and implantation. To accomplish this gentle transfer, an improved catheter (referred alternatively and interchangeably herein as “microcatheter”) with a beveled opening and tip is described. The catheter is able to work as both a microsurgical instrument, used in a method described herein to form an embryo-receiving pocket within the endometrial lining of a subject's uterus, and as the vehicle for transferring an embryo into the pocket. It has been observed that by gently securing an embryo within a pocket of endometrial lining, many of the risks of IVF, such as a tubal pregnancy, misplacement of the embryo, and loss of the embryo can be minimized. Tubal pregnancies, for example, are virtually eliminated according to this method.

### BRIEF DESCRIPTION OF THE DRAWINGS

FIG. 1 is a side view of an embodiment of a catheter or microcatheter.

FIG. 2 is a side view of the distal end of the microcatheter of FIG. 1.

FIG. 3 is a perspective top side view of the distal end of the microcatheter of FIG. 1.

FIG. 4 is a schematic, cross-sectional side view of an embodiment of a hysteroscope.

FIG. 5 is a cross-section side view of a distal end of the microcatheter of FIG. 1 containing an embryo for implantation.

FIG. 6 is a first sequential view of an embodiment of a method of assisted embryo implantation, which shows the survey of the endometrial lining for an implantation site.

FIG. 7 is a second sequential view of the method of assisted embryo implantation, which shows the formation of an embryo-receiving pocket at the selected implantation site.

FIG. 8 is a third sequential view of the method of assisted embryo implantation, which shows the implantation of the embryo within the pocket of FIG. 7.

FIG. 9 is a fourth sequential view of the method of assisted embryo implantation, which shows the closure of the embryo-receiving pocket over the embryo.

### DETAILED DESCRIPTION

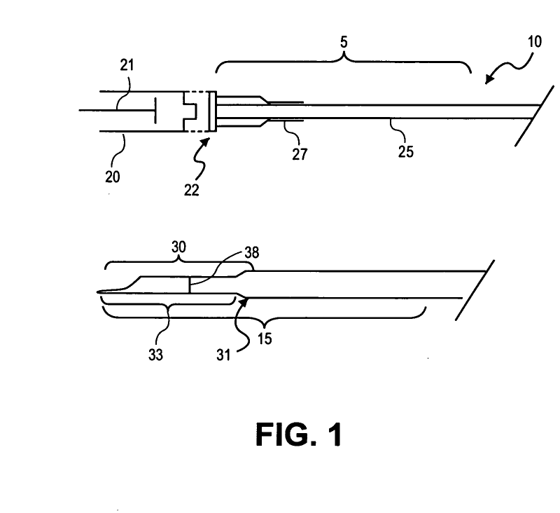

Referring now to the drawings, illustrated in FIGS. 1-3 is one embodiment of a microcatheter. Microcatheter 10 includes, in this embodiment, proximal portion 5 and distal 15. Microcatheter 10 includes shaft or cannula 25 having a lumen therethrough. Shaft 25 terminates at distal shaped end 30. Proximal portion 5 includes, at proximal end 22, a hub to mate with operational syringe 20, with plunger 21. The hub is, for example, a luer lock fitting. In one embodiment, extending approximately 25-30 millimeters from the hub is stabilizer 27 of, for example, a polymer tube having an inner diameter slightly greater than an external diameter of shaft 25.

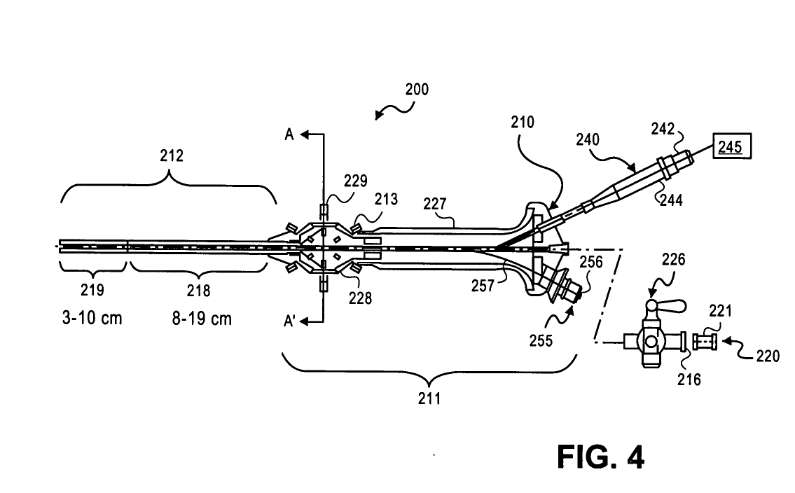

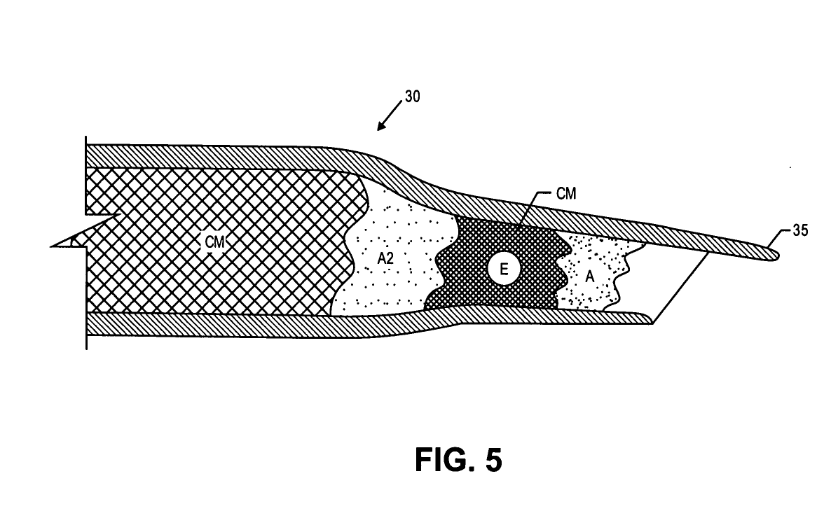

Shaft 25 defines a lumen therethrough for, representatively, introducing one or more embryos into a uterus of a subject. In one embodiment, shaft 25 is an extruded one piece polymer material having a length on the order of 70 centimeters (cm). Suitable polymers for shaft 25 are selected such that the shaft has sufficient rigidity to be advanced through an endoscope, specifically through an endoscopic cap inserted in an endoscope (see, e.g., endoscopic cap 221 in FIG. 4) to penetrate the endometrial lining of a subject's uterus (see, e.g., FIGS. 5-9 and the accompanying text). The polymer material is also selected such that shaft 25 is flexible enough so the shaft does not penetrate the uterine muscle of the subject. One suitable polymer is polycarbonate (e.g., transparent polycarbonates). Tetrafluoroethylene (e.g., TEFLON™), polyurethane, polyethylene, and nylon materials may also be suitable. A suitable outside diameter for a proximal portion of shaft 25 is on the order of one millimeter (mm) or less. Shaft 25 includes a distal portion including shaped end 30. An external marking (e.g., marking 38) may be included at a position, for example, one centimeter (cm) from the distal end of shaft 25 to provide a visual identification of either the volume of contents within microcatheter 10 or a location of microcatheter 10, for example, in tissue. One way to form marking 38 is by placing a ring-shaped heat shrink on shaped end 30 and thermally bonding the ring to the shaft.

Shaped end 30 of microcatheter 10 includes base region 31 of a similar diameter as the flexible hollow shaft 25 (e.g., 1 mm or less) and then tapers over 1 to 3 mm into narrow distal end 33 which is, for example, approximately one to two centimeters in length, with a representative outside diameter of 0.8 mm or less (e.g., an outside diameter less than the outside diameter of a non-tapered portion of the shaft). In one embodiment, distal end 33 has an interior diameter of approximately 10 micrometers (μm) or larger, preferably between 400 to 500 μm.

Microcatheter 10 also includes angled or beveled opening 34 at its distal end. Beveled opening 34 extends between first point 40 that defines a length of shaft 25 and second point 45 at a distal end of the opening. Angle, γ, of beveled opening 34 between a projection including first point 40 and second point 45 and a perpendicular projection from point 45 of shaft 25 is 0° to 45°. A representative length, L₁, of beveled opening 34 is 0.1 to 1.5 millimeters (mm).

Opening 34 may be formed only between first point 40 and second point 45 or extend into portion 35 as shown in FIG. 3.

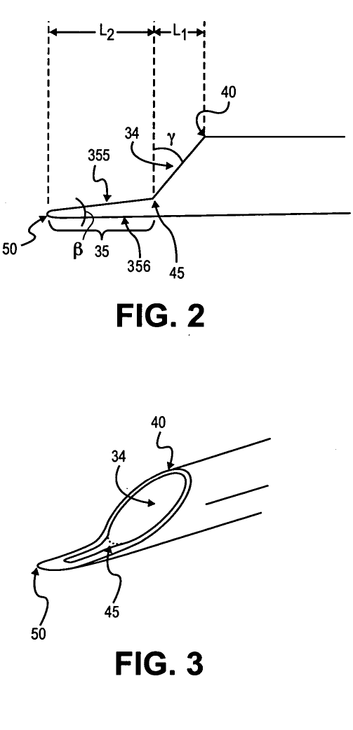

Extending distally from second point 45 is tip 35. FIG. 2 illustrates a sharp transition between beveled opening 34 and tip 35. It is appreciated that the transition may be gradual (e.g., curved).

Surface 355 of tip 35 (on the side of beveled opening 34) may have an angle, β, relative to opposite surface 356 of 0° to 45° and a length, L₂, on the order of 0.1 mm to 3 mm. In one embodiment, tip 35 is part of the polymer body of shaft 25. To form tip 35, a polymer tube may be cut to a length that includes tip 35. Beveled opening 35 may then be formed by a second proximal cut that does not extend completely through the tube. The portion of tubing extending distally from beveled opening 34 (from second point 45) may then be trimmed into an arrow-like shape to form tip 35 (e.g., with a tip or point defining the distal end).

Opening 34 is the vehicle through which an embryo is delivered into the implantation site and may also be the microsurgical instrument used to form an implantation pocket within the endometrial lining as described with reference to FIGS. 5-9 and the accompanying text. A point at the distal end of shaft 25 representing the greatest length of shaft 25 defines tip 35. A portion of the body of shaft 25 including tip 35 may be beveled in a direction opposite bevel angle γ to yield a more refined cutting tool.

FIG. 4 shows a schematic, cross-sectional view of an embodiment of a hysteroscope. In this embodiment, hysteroscope 200 includes operational section 211 at one end (a proximal end) and hybrid insertion arm 212 at a second end (a distal end). Hybrid insertion arm 212 is generally tubular (defining one or more lumens therethrough) and includes proximal portion 218 of a generally rigid material, such as stainless steel or a rigid polymer material, and distal portion 219 of a relatively flexible material (e.g., a polymer material such as polycarbonate or polyethylene). Representatively, proximal portion 218 has a length on the order of about 5 to 30 centimeters (cm) with about an outside diameter (OD) on the order of 3 to 4 mm. Distal portion 219 has a representative length of 3 to 15 cm and a representative OD of 2.5 to 4 mm, preferably 3.0 to 3.5 mm, and preferably a representative diameter slightly smaller (at least toward distal end 230) than proximal portion 218.

Referring to FIG. 4, operational section 211 includes handle portion 227 that is preferably knurled for better holding and feel. Coupled to a distal end of handle portion 227 is lever holder 228. Disposed within lever holder 228 is articulating lever 229 that is coupled through, for example, wire members (e.g., braided wire members) to distal portion 229. Representatively, deflection of articulating lever 229 about lever holder 228 deflects distal portion 219 of hybrid insertion arm 212 to the same degree. In one embodiment, articulating lever 229 rotates about a single axis 60° in two directions (e.g., clockwise and counterclockwise) for a total range of deflection of 120°. Protruding stops 213 on lever holder 228 may be included to limit articulation of articulating lever 229.

Referring to FIG. 4, at a proximal end of handle portion 227 of hysteroscope 200 is access port 216. Access port 216 provides access to operational channel or lumen 220. Operational channel 220 extends through the device from operational section 211 to hybrid insertion arm 212 terminating at distal tip 230. In this embodiment, access port 216 is axially aligned with operational channel 220. In one regard, the axial alignment aids the insertion of instruments such as a microcatheter into operational channel 220.

In some embodiments, a microcatheter or other instrument may be inserted in operational channel 220 through access port 216 at the same time as a gas or fluid is administered through the hysteroscope to a patient. To minimize leakage of gas or fluid around a microcatheter (e.g., microcatheter 10) or other instrument, endoscopic cap 221 is placed in access port 216. Endoscopic cap 221 of an elastic material has an opening therethrough to allow access to operational channel 220. In one procedure, endoscopic cap 221 is fitted into access port 216 and a blunt needle (e.g., an 18 gauge needle) having a lumen of a diameter suitable to allow the passing of a microcatheter or other instrument therethrough is inserted through endoscopic cap 226. The microcatheter or other instrument is then inserted through the blunt needle and advanced into operational channel 220 as desired. Once the microcatheter or other instrument is positioned, the blunt needle may be removed.

Also at a proximal end of handle portion 227 of hysteroscope 200 is a portion of illumination train 240 including illumination holder 244. A plurality of illumination fibers (e.g., glass fibers) are disposed within illumination holder 244 and join operational channel 220 within handle 227.

At a proximal end of handle 227 is a portion of image train 255 including eyepiece 256. Eyepiece 256 is coupled to lumen 236 (see FIGS. 9 and 10) which joins operational channel 220 within handle 227 and is axially aligned within a primary lumen extending from operational section 211 to hybrid insertion arm 212.

Coupled at a proximal end of operational channel 220 is valve 226 to, in one position, seal or block operational channel 220 and, in another position, to allow insufflation gas or an instrument such as a microcatheter to be passed through operational channel 220. In another embodiment, valve 226 may have three positions to, for example, provide individual access ports for an instrument and for gas or fluid (e.g., allowing introduction of a gas or fluid through operational channel 220 at the same time an instrument is inserted through operational channel 220). In one embodiment, valve 226 includes a positioning portion that may be handled by an operator to position valve 226 and that is sterilizable, removable and replaceable. A microcatheter and/or insufflation gas, in one embodiment, may alternatively be introduced to operational channel 220 at entry port 216.

FIGS. 5-9 show the sequential performance of an embryo implantation procedure representatively using microcatheter 10 and hysteroscope 200. The biology, timing and biochemistry involved in embryo selection and in optimizing the subject for implantation is not the topic of this invention. It is well known by those skilled in the art of how best to harvest and fertilize eggs and how best to select viable embryos. Volumes of scientific literature also exists on the hormonal, pharmaceutical and other chemical factors which should be orchestrated, monitored and taken into account when selecting the timing for embryo implantation. Accordingly, such information is omitted.

Prior to any intrauterine activity, an embryo must be placed in microcatheter 10. Microcatheter 10 will be used to both prepare the site for implantation and to transfer the embryo “E” into the site. Shown in FIG. 5 is an embryo “E” immersed in a culture medium “CM” placed near distal end 33 of microcatheter 10. The culture medium “CM” serves the important role of maintaining the health and viability of the embryo “E” during the procedure. In this embodiment, the culture medium “CM” used is a “modified Human Tubal Fluid” manufactured by Irvine Scientific of Irvine, Calif. Considering the rapid pace of advancements in IVF, new and varied culture media will undoubtedly be developed or become available. Accordingly, the method described should not be limited to that culture media described herein, but rather to any suitable culture media which serves the function of maintaining embryo viability during the implantation procedure.

Prior to placing the embryo “E” into microcatheter 10, a first quantity of culture medium “CM” is drawn into microcatheter 10 and followed by a back measure of atmospheric air “A2” (e.g., 10-20 microliters (μL)). Next, the embryo “E”, bathed in more culture medium “CM” (e.g., 5-10 μL), is drawn into distal end 33 of microcatheter 10 followed by a front measure of atmosphere air “A” (e.g., 5-10 μL), thereby sandwiching the embryo “E” between a first and second measure of atmospheric air “A” and “A2”. Once loaded with the embryo “E”, microcatheter 10 is ready for use in the implantation procedure. Each measure of atmospheric air may be, for example, about three to twenty microliters in volume.

In one procedure, endoscopic cap 221 is inserted into access port 216 of hysteroscope 200 (see FIG. 4). A blunt needle having a lumen of a diameter suitable to allow the passing of microcatheter 10 therethrough, is inserted through endoscopic cap 221. Microcatheter 10 loaded as described above is threaded into operational channel 220 of hysteroscope 200, so that tip 35 is approximately one to two centimeters (cm) from distal end 230. The blunt needle may then be removed from the endoscopic cap so that the cap snugly surrounds microcatheter 10.

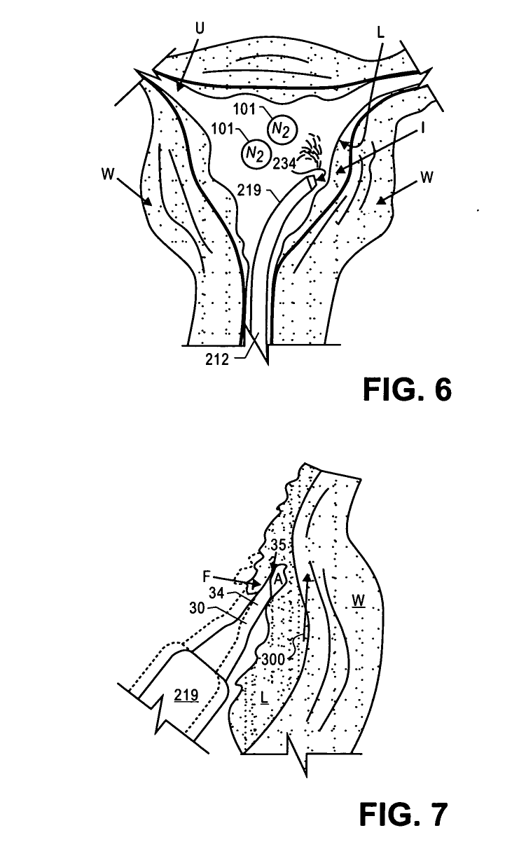

Distal portion 212 of representatively hysteroscope 200 is guided into the uterus “U” (FIG. 6). During the insertion of the hysteroscope 200, N₂ gas 101 is fed into the uterus “U” pressurizing or insufflating the uterus “U” and thereby distending the uterine walls “W”. Depending on the needs of the operator, and the uterus of the subject, the gas 101 may be automatically maintained at a constant pressure or the operator may vary the pressure. The distension of the uterine walls “W” enhances the visualization through hysteroscope 200 within the uterus “U”.

Once an embryo implantation site “I” is selected, microcatheter 10 is inserted into the endometrial lining “L” (FIG. 7) with tip 35 moved generally along the path of arrow 300 making a small incision two to five millimeters (mm) deep in the endometrial lining “L” to form a small flap “F”. The front measure of atmospheric air “A” is then released from microcatheter 30 and acts to lift up the small flap “F” of the endometrial lining “L”.

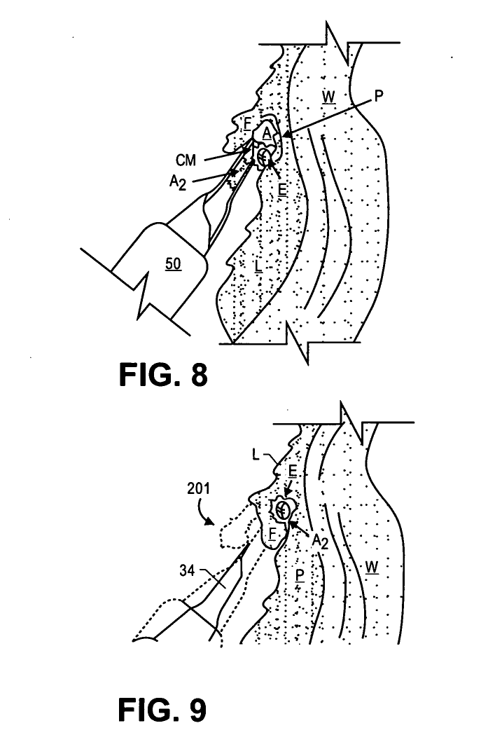

Shown in FIG. 8 is the embryo-receiving pocket “P” formed beneath the small flap “F”. The actual implantation of the embryo “E” into the embryo-receiving pocket “P” is performed with the same microcatheter 30 used to form the embryo-receiving pocket “P” and is accomplished by depressing plunger 21 of syringe 20 (see FIG. 1) to gently urge the embryo “E” and the back measure of atmospheric air “A2” out of microcatheter 30 and into embryo-receiving pocket “P”.

The back measure atmospheric air “A2” forms a cushion around the embryo “E” which helps to protect it when the microcatheter is removed (FIG. 9) and the small flap “F” drops back into place over the embryo “E” along the line of arrow 201. To complete the procedure, hysteroscope 200 is then gently removed from the subject and post-IVF precautions and protocols should be used. Another possible advantage of a successful implantation of the embryo “E” within the endometrial lining “L” is that the length of the post-IVF precautions may be reduced.

Dependent on the subject, the number of viable embryos available and the aperture, up to two embryos may be implanted into a single pocket “P”. In the case of embryo implantations into multiple pockets, additional embryos, each bathed in culture medium, are sandwiched between a measure of atmospheric air within the microcatheters and implanted into separately formed pockets “P”.

Certain presently preferred embodiments of apparatus and methods for practicing the invention have been described herein in some detail and some potential modifications and additions have been suggested. Other modifications, improvements and additions not described in this document may also be made without departing from the principles of the invention. For example, the microcatheter (e.g., microcatheter 10) and hysteroscope (e.g., hysteroscope 200) have been described with reference to an IVF procedure. It is appreciated that such devices need not be specified together and either may have other uses beyond IVF procedures. Representatively, the hysteroscope may be used in connection with other devices such as biopsy forceps or other procedures such as irrigation/aspiration. The microcatheter and hysteroscope (end) are also contemplated in other than intrauterine procedures. One non-limiting example would be gastroenterological procedures.

I claim:

1. A catheter comprising:
a shaft comprising a body with a proximal portion and a distal portion, the body defining an opening from the proximal portion to the distal portion, the distal portion having an exterior dimension suitable for insertion into a body of a subject as a procedural instrument, the distal portion having an end that is beveled in a first direction across an end opening, such that a length of the shaft to a first point is a first length and a length of the shaft to a second point on the end is a second length longer than the first length, a portion of the shaft extending from the second point defining a tip, wherein the tip comprises a material that has sufficient rigidity to penetrate an endometrial lining of a subject and sufficient flexibility to resist penetration of a uterine muscle of a subject.

2. The catheter of claim 1, wherein the beveled end defines an angle of 0° to 45° between a projection including the first point and the second point and a perpendicular projection from the second point.

3. The catheter of claim 1, wherein tip has a length of 0.1 millimeters to 3 millimeters.

4. The catheter of claim 1, wherein the tip comprises an arrow-like shape.

5. The catheter of claim 1, wherein the distal portion of the body comprises a first portion having a first outside diameter and a second portion distal to the first portion having a second outside diameter that is less than the first outside diameter.
